(19) United States
(12) Patent Application Publication
(10) Pub. No.: US 2015/0087959 A1
(43) Pub. Date: Mar. 26, 2015

(54) Correction of Capacitively Acquired EKG Signals in Conjunction with Measurements

(71) Applicant: Jürgen Rößler, Erlangen (DE)

(72) Inventor: Jürgen Rößler, Erlangen (DE)

(21) Appl. No.: 14/486,567

(22) Filed: Sep. 15, 2014

(30) Foreign Application Priority Data
Sep. 24, 2013 (DE) 102013219117.0

(51) Int. Cl. A61B 5/00; A61B 5/04; A61B 5/0408; A61B 6/03; A61B 6/04; A61B 5/0432; A61B 5/0205; A61B 5/044

(52) U.S. Cl. CPC A61B 5/7217; A61B 5/0205; A61B 5/04012; A61B 5/044; A61B 6/032; A61B 6/0407; A61B 5/0432; A61B 5/0408; A61B 5/0555
USPC 600/411; 600/427; 600/407

(57) ABSTRACT

An interference value that occurs during a measurement using a medical imaging examination device is determined. At least one EKG signal is measured using a capacitive EKG sensor during a measurement using the medical imaging examination device. The measured EKG signal is corrected using the determined interference value. The corrected EKG signal is displayed or stored.

### RELATED CASE

This application claims the benefit of DE 102013219117.0, filed on Sep. 24, 2013, which is hereby incorporated by reference in its entirety.

### TECHNICAL FIELD

The present embodiments relate to correcting capacitively acquired EKG signals in conjunction with measurements using a medical imaging examination device.

### BACKGROUND

EKG measuring apparatuses are primarily used to measure and monitor the cardiac function of a patient. The total voltage of the electrical activity of the cardiac muscle fibers is typically measured as a so-called “EKG signal” by way of at least two electrodes adhered to the body of the patient to be examined.

There are other applications as well as simply monitoring the cardiac function of a patient. For example, EKG signals are also used in medical imaging to generate trigger signals. During imaging, information about the cardiac phase is obtained by way of the EKG signal, in order, thus, to synchronize imaging with cardiac activity. It is thus possible to take high-quality recordings of the heart or recordings of regions moved by the heartbeat, in particular, using imaging methods that have quite long recording times.

EKG measuring apparatuses are also used for the in-situ acquisition of EKG signals while a patient is being examined using a magnetic resonance device or a computed tomography device. The determination of R waves in EKG signals is essential for reliable triggering. However, with magnetic resonance devices, interference results, for example, from T wave increases occurring in the magnetic field and other interference coupled into the EKG signals measured with the EKG measuring apparatuses. The interference is due to the powerful gradient fields and high-frequency fields used for imaging therein. In the case of computed tomography devices, interference is produced in the measured EKG signals by gantry rotation.

Such interference is extremely undesirable. To synchronize the recording of a magnetic resonance image with the heartbeat, the R wave of the EKG signal is identified reliably. Interference signals may be wrongly interpreted as an R wave, for example, due to their often similar shape. The triggering of a recording of a magnetic resonance image or a computed tomography image is thereby incorrectly initiated. On the other hand, a “true” R wave is not identified as such due to the overlaid interference signals. This regularly causes a significant deterioration in image quality.

When a magnetic resonance device is used as a medical imaging examination device, it is known, for example from the article by Odill et al., “Noise Cancellation Signal Processing Method and Computer System for Improved Real-Time Electrocardiogram Artifact Correction during MRI Data Acquisition”, IEEE Transactions on Biomedical Engineering, Vol. 54, No. 4, April 2007, or the article by Felblinger et al., “Restoration of Electrophysiological Signals Distorted by Inductive Effects of Magnetic Field Gradients During MR Sequences”, Magnetic Resonance in Medicine 41, pp 715-721 (1999), that such interference may be estimated and the measured EKG signals corrected accordingly.

The correct application of the commercially available adhesive EKG electrodes is complex and not particularly pleasant for the patient because of the gel that has to be used.

This problem would not exist with capacitive EKG sensors which measure cardiac signals contactlessly. However capacitive EKG sensors are even more sensitive to interference due to changing electrical and magnetic fields than conventional adhesive EKG sensors. Capacitive EKG sensors are known, for example, from the article “Berührungslose EKG-Messungen mit EPS/Elektro-Potential-Sensoren and Zusatzanwendungen” (Contactless EKG measurements using EPS/electropotential sensors and additional applications) meditronix-journal February 2012, pp 26-27.

### SUMMARY AND DESCRIPTION

The scope of the present invention is defined solely by the appended claims and is not affected to any degree by the statements within this summary.

EKG measurements for triggering use capacitive EKG sensors in conjunction with medical imaging examination devices, which generate changing electrical and/or magnetic fields in the region of the EKG measurement.

One embodiment of a method for correcting capacitively acquired EKG signals in conjunction with a medical imaging examination device includes the following acts:
determining an interference value that occurs during a measurement using the medical imaging examination device,
measuring at least one EKG signal using a capacitive EKG sensor during a measurement using the medical imaging examination device,
correcting the measured EKG signal using the determined interference value, and
displaying and/or storing the corrected EKG signal.

By determining interference values during a measurement using the medical imaging examination device, it is possible to correct further EKG signals measured using the capacitive EKG sensor. The further EKG signals are measured during a further measurement using the medical imaging examination device, and thus eliminate the coupling-in of interference as a result of measurement using the medical imaging examination device therefrom, as the determined interference values also allow coupled-in interference to be identified more easily in sensitive contactless capacitive EKG sensors. The identified coupled in interference is suppressed. EKG signals corrected in this manner may be used, for example, to determine R waves in the acquired EKG signals, by searching for R waves in the corrected EKG signals. The correction of an EKG signal allows reliable and accurate identification of R waves even in EKG signals acquired using a capacitive EKG sensor, despite ambient interference due to the medical imaging examination device. When an R wave is successfully detected in the corrected EKG signal, the detection may be used to trigger further measurements using the medical imaging examination device, in that a trigger signal is output to the medical imaging examination device. The corrected EKG signals therefore allow accurate triggering of the recording of medical image data using a medical imaging examination device and thus contribute to the good image quality of such recordings, such as recordings of the heart.

An EKG measuring apparatus includes at least one capacitive EKG sensor, a processing unit, a computation unit and a storage unit, which interact in such a manner that the method may be performed.

A medical imaging examination device includes at least one control unit and an EKG measuring apparatus with at least one capacitive EKG sensor, a processing unit, a computation unit and a storage unit, which interact in such a manner that the method may be performed. The corrected EKG signal may be used, for example, to determine trigger signals for triggering measurements using the medical imaging examination device. The capacitive EKG sensor may be integrated, for example, in an examination couch of the medical imaging examination device.

The advantages and details cited in relation to method also apply in a similar manner to the EKG measuring apparatus and the medical imaging examination device.

Further advantages and details of the present invention may emerge from the exemplary embodiments described in the following and with reference to the drawings. The cited examples do not restrict the invention in any manner.

### DESCRIPTION OF THE ACCOMPANYING DRAWINGS

FIG. 1 shows a schematic diagram of one embodiment of an EKG measuring apparatus by way of example in conjunction with a medical imaging examination device,

FIG. 2 shows a flow diagram of one embodiment of a method for correcting capacitively acquired EKG signals in conjunction with a medical imaging examination device.

### DETAILED DESCRIPTION OF THE PREFERRED EMBODIMENTS

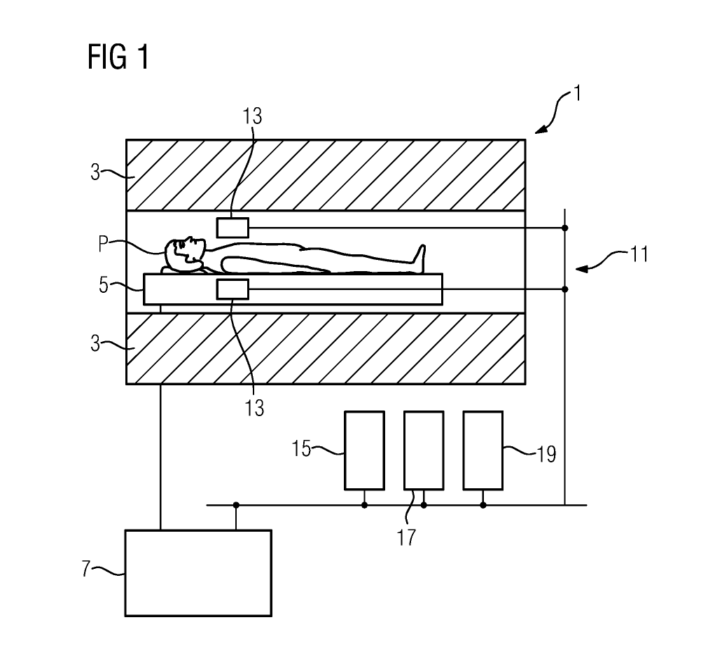

FIG. 1 shows a schematic diagram of an EKG measuring apparatus 11 with at least one capacitive EKG sensor 13. The capacitive EKG sensor 13 measures EKG signals of a patient P without direct skin contact. To this end, the at least one capacitive EKG sensor 13 may be positioned contactlessly in the vicinity of the patient P or may even be integrated in a component 5 of a medical imaging examination device 1. The EKG sensor 13 may, for example, be integrated in an examination couch, which is at least partially enclosed by a housing 3 of the medical imaging examination device 1 during a measurement. The EKG sensor 13 may be integrated in a fixing apparatus or, when a magnetic resonance device is being used as a medical imaging examination device 1, in a local coil. In one exemplary embodiment, the EKG measuring apparatus 11 includes not only the at least one contactless capacitive EKG sensor 13 but also at least one conventional EKG sensor, which is to be adhered to the skin of the patient P. With such an embodiment, it is possible at least to facilitate the preparation of the patient P with EKG sensors.

In one exemplary embodiment, the EKG measuring apparatus 11 includes precisely two such capacitive EKG sensors 13. The electrical signal of the heart of a patient P may be detected with sufficient accuracy using just two contactless EKG sensors.

In a further exemplary embodiment, the EKG measuring apparatus 11 includes at least three such capacitive EKG sensors 13. Using three or more EKG sensors 13, it is possible to improve the quality of the measured EKG signal even further, for example by further processing (e.g., averaging and/or by determining reference signals and the like).

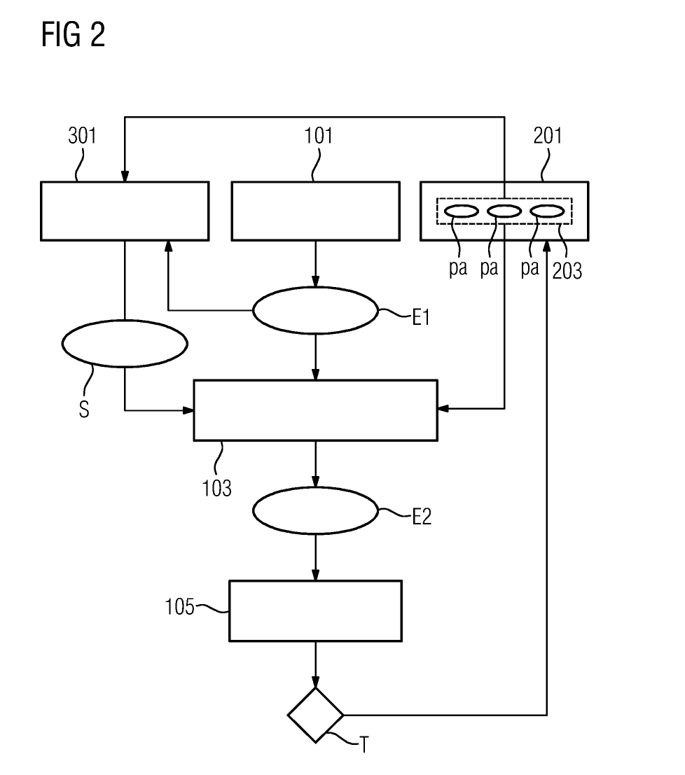

The EKG measuring apparatus 11 also includes a processing unit 15, a computation unit 17 and a storage unit 19, which interact in such a manner that a method, as described in more detail with reference to FIG. 2, may be performed. The separate or composite representation of these units is not necessarily physical but should rather be seen as a separation or combination based on notional units. The EKG measuring apparatus 11, the processing unit 15, the computation unit 17 and the storage unit 19 are connected to the medical imaging examination device 1, in particular a control unit 7 of the medical imaging examination device 1, and to one another for the transfer of data. The control unit 7 of the medical imaging examination device 1, in particular, controls measurements using the medical imaging examination device and sets the required parameters for the respective measurement.

FIG. 2 shows a schematic flow diagram of a method. In an act 301, an interference value S that occurs during a measurement 201 using the medical imaging examination device 1 is determined.

In an act 101, at least one EKG signal E1 is also measured using a capacitive EKG sensor 13, while a measurement 201 is being performed using the medical imaging examination device 1.

In an act 103, the measured EKG signal E1 is corrected using the determined interference value S. For example, the EKG signal E1 measured using the capacitive EKG sensor 13 may be corrected by subtracting the determined interference value S from the measured EKG signal E1: E2=E1−S.

In a further act 105, the corrected EKG signal E2 may then be displayed and/or stored and/or further processed.

For example, the EKG signals measured by the EKG electrodes 13 of the EKG measuring apparatus 11 are supplied to the processing unit 15 for electronic processing.

It is possible to calculate the interference value using at least one parameter pa from a parameter set 203 used for a current measurement 201 and optionally the current interference value from already stored interference values and EKG signals E1 in the computation unit 17. The measured EKG signals E1 now available electronically may also be corrected according to the proposed method, and corrected EKG signals E2 may be examined, for example, for the occurrence of R waves. Measured and/or corrected EKG signals and intermediate processing results and further parameters, in particular parameters pa, which were used to correct the measured EKG signals, may be stored in the storage unit 19. When an R wave is detected (T) based on a corrected EKG signal E2, a trigger signal triggering a further measurement 201 using the medical imaging examination device 1 can be dismissed.

The determination of the interference value S in act 301 may be estimated by an interference estimation method based on the parameters defined for measurement using the medical imaging examination device.

When a magnetic resonance device is used as a medical imaging examination device 1, the interference value S may be determined from the gradients switched during the magnetic resonance measurement 201 as parameters pa, by estimating the pulse responses generated by the switched gradients. This allows the interference value S to be determined from: S=h_Ix_U(t)*Ix(t)+h_Iy_U(t)*Iy(t)+h_Iz_U(t)*Iz(t), where “*” is the convolution operator and Ix(t), Iy(t) and Iz(t) are the currents injected into the respective gradient coils and h_Ix_U(t), h_Iy_U(t) and h_Iz_U(t) are the cited pulse responses, which represent the coupling in characteristic of the respective gradient coil into the EKG signal E1 measured at the capacitive EKG sensor 13.

The pulse responses h_Ix_U(t), h_Iy_U(t) and h_Iz_U(t) may be determined for this purpose, for example, by defined test measurements before the actual measurement 201. In this process, a test EKG signal E1, for example, may be measured during a test measurement 201. During a test measurement 201, for example, just one gradient of the magnetic resonance device 1 may be switched in each instance to favor generalization to other gradient switches and to facilitate subsequent extrapolation to any gradient switches.

If test EKG signals E1 are measured in this manner for each gradient coil (x, y, z) present and stored together with the parameters pa used for each of the test measurements 201, the pulse responses may be extracted from the test EKG signals.

In this process, the actual “interfering” cardiac signal contained in the test EKG signal may then be suppressed by performing a test measurement a number of times, for example, and taking the average of the associated test EKG signals.

Pulse responses and thus interference values for any parameters pa may be determined adaptively from the test EKG signals thus obtained for defined parameters pa.

When a computed tomography device is used as a medical imaging examination device 1, the interference value may be determined using a similar linear estimation method, by estimating the pulse response to gantry rotation as a parameter pa. The interference value S here is obtained from:

S=h_{—}R_{—}U(t)*R(t),

where “*” is the convolution operator again, R(t) is the angular position of the gantry and h_R_U(t) is the pulse response measured due to the excitation R(t) as the signal in the capacitive EKG sensor 13.

The pulse response h_R_U(t) here may be measured, for example, before the actual CT measurement 201 by a test rotation of the gantry and measuring the test EKG signal E1 occurring in this process at the capacitive sensor 13. Again, the actual “interfering” cardiac signal contained in this instance in the test EKG signal may be suppressed, by performing a test rotation a number of times, for example, and taking the average of the associated test EKG signals. Further variables influencing the measured EKG signal E1, in addition to gantry rotation, may be treated in a similar manner, with the above equation being extended accordingly.

Such a test measurement and measurement of test EKG signals before the actual measurement 201 mean that the determined interference value is particularly well adjusted to actual conditions and may thus correct the EKG signal E1, which was measured during the actual measurement 201 and has to be corrected, particularly efficiently.

It is however also conceivable to perform test measurements for different initial angular positions of the gantry and different gantry speeds and to store the associated test EKG signals and extract a current interference value S adaptively from the stored test EKG signals during subsequent actual measurements.

Generally at least one interference value may be determined in each instance and stored in relation to the parameters used for the respective measurement using the medical imaging examination device for different measurements. By creating such a “parameter/interference table,” it is possible to determine the current interference value even more accurately during subsequent measurements, by, for example, performing plausibility checks based on the “parameter/interference table”. In some circumstances, if the “parameter/interference table” is adequately populated, the entire determination of the interference values may take place in the manner of an adaptive determination based on said “parameter/interference table”, allowing test measurements to be dispensed with.

An optionally variable couch position of an examination couch used for measurement 201 using the medical imaging examination device may also be included as a further parameter for determining the interference value in the determination of the interference value S. To this end, the abovementioned test measurements 201, for example, are also performed as a function of a couch position of the examination couch used. If a sufficient number of test measurements are performed, e.g. for the three individual gradient coils in the case of a magnetic resonance device, in a sufficient number of couch positions and the associated test EKG signals are stored, interference signals may be determined for any further measurements 201 based on the stored test EKG signals and subject to adaptive adjustment of the parameters of the test measurements to the parameters of the current measurement 201, without a new test measurement having to be performed, thereby reducing delays in measuring mode. For example, test EKG signals measured once for a magnetic resonance device and pulse responses determined therefrom may then be used in conjunction with the current couch position for any further MR measurements to determine the interference value. Similarly, test EKG signals measured once for a computed tomography device and pulse responses determined therefrom may then be used in conjunction with the current couch position for any further CT measurements to determine the interference value.

Relevant parameters pa from the parameter set 203 used during the measurement 201 using the medical imaging examination device 1 may thus be at least one parameter from the group of parameters consisting of a couch position of the examination couch used during the measurement, if the medical imaging examination device is a magnetic resonance device, the gradients switched during the measurement and, if the medical imaging examination device is a computed tomography device, a rotation or angular position of the gantry at the start of the measurement.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims can, alternatively, be made to depend in the alternative from any preceding or following claim, whether independent or dependent, and that such new combinations are to be understood as forming a part of the present specification.

While the present invention has been described above by reference to various embodiments, it should be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for correcting capacitively acquired EKG signals in conjunction with a medical imaging examination device, the method comprising:
determining an interference value that occurs during a measurement using the medical imaging examination device,
measuring at least one EKG signal using a capacitive EKG sensor during a measurement using the medical imaging examination device,
correcting the measured EKG signal using the determined interference value, and
displaying and/or storing the corrected EKG signal.

2. The method as claimed in claim 1, wherein the determination of an interference value is estimated based on the parameters defined for measurement using the medical imaging examination device.

3. The method as claimed in claim 1, wherein determining comprises determining at least one measurement of a test EKG signal during at least one test measurement using the medical imaging examination device.

4. The method as claimed in claim 3, wherein determining comprises an adaptive determination based on at least one already measured test EKG signal.

5. The method as claimed in claim 3, wherein the parameters for a test measurement using the medical imaging examination device are selected in such a manner that generalization to other possible parameters is favored.

6. The method as claimed in claim 1, wherein at least one interference value is determined in each instance and stored in relation to the parameters used for the respective measurement using the medical imaging examination device for different measurements using the medical imaging examination device.

7. The method as claimed in claim 6, wherein the determination of an interference value comprises an adaptive determination based on at least one already stored interference value taking into account at least one parameter of the measurement performed using the medical imaging examination device during the measurement of the current EKG signal.

8. The method as claimed in claim 1, wherein the relevant parameters used during the measurement using the medical imaging examination device comprise at least one parameter from the group of parameters consisting of a couch position of the examination couch used during the measurement, if the medical imaging examination device is a magnetic resonance device, the gradients switched during the measurement and, if the medical imaging examination device is a computed tomography device, a rotation of the gantry at the start of the measurement.

9. The method as claimed in claim 2, wherein determining comprises determining at least one measurement of a test EKG signal during at least one test measurement using the medical imaging examination device.

10. The method as claimed in claim 4, wherein the parameters for a test measurement using the medical imaging examination device are selected in such a manner that generalization to other possible parameters is favored.

11. The method as claimed in claim 5, wherein at least one interference value is determined in each instance and stored in relation to the parameters used for the respective measurement using the medical imaging examination device for different measurements using the medical imaging examination device.

12. The method as claimed in claim 7, wherein the relevant parameters used during the measurement using the medical imaging examination device comprise at least one parameter from the group of parameters consisting of a couch position of the examination couch used during the measurement, if the medical imaging examination device is a magnetic resonance device, the gradients switched during the measurement and, if the medical imaging examination device is a computed tomography device, a rotation of the gantry at the start of the measurement.

13. An EKG measuring apparatus comprising:
at least one capacitive EKG sensor,
a processing unit,
a computation unit, and
a storage unit,
wherein the processing unit, the computation unit or the processing unit and computation unit are configured to determine an interference value that occurs during a measurement using a medical imaging examination device, measure at least one EKG signal using the at least one capacitive EKG sensor during a measurement using the medical imaging examination device, correct the measured EKG signal using the determined interference value, and display and/or store the corrected EKG signal in the storage unit.

14. A medical imaging examination device comprising:
a control unit; and
an EKG measuring apparatus with at least one capacitive EKG sensor, a processing unit, a computation unit and a storage unit, wherein the processing unit, the computation unit or the processing unit and computation unit are configured to determine an interference value that occurs during a measurement using a medical imaging examination device, measure at least one EKG signal using the at least one capacitive EKG sensor during a measurement using the medical imaging examination device, correct the measured EKG signal using the determined interference value, and display and/or store the corrected EKG signal in the storage unit.

15. The medical imaging examination device of claim 14 wherein the control unit is configured to use the corrected EKG signal to determine trigger signals for triggering measurements using the medical imaging examination device.

16. The medical imaging examination device as claimed in claim 14, wherein the capacitive EKG sensor is integrated in a component of the medical imaging examination device.
